(19) United States
(12) Patent Application Publication
(10) Pub. No.: US 2003/0135262 A1
(43) Pub. Date: Jul. 17, 2003

(54) Apparatus for piezo-electric reduction of concretions

(76) Inventors: Stephen P. Dretler, Wayland, MA (US); Gerald L. Wilson, Wayland, MA (US)

Correspondence Address: McCormick, Paulding & Huber, City Place II, 185 Asylum Street, Hartford, CT, 06103-3402, US

(21) Appl. No.: 10/050,321

(22) Filed: Jan. 15, 2002

(51) Int. Cl. A61F 2/06

(52) U.S. Cl. 623/1.15; 606/32

(57) ABSTRACT

A piezo-electric stent and capillary tube is disclosed wherein the polarized piezoelectric material is cylindrical and is disposed between two concentric cylindrical electrodes. The piezo-electric material is polarized either radially or uniformly perpendicularly to the axis of the stent. The stent or capillary tube is provided with leads to each electrode that may then be connected to a stationary or portable energy source. The piezo-electric device of the invention is useful to reduce concretions forming on the stent and to reduce the size of kidney stones or other bodily concretions. The invention produces vibrations which themselves may be beyond the yield strength of the target concretion or have a high cycle frequency which fatigues concretions to the point of failure of the concretion.

### BACKGROUND OF THE INVENTION

1. Field of the Invention

The invention relates to medical instruments for in situ treatment of concretions. More particularly, the invention relates to piezo-electric stents and capillary tubes for in situ reduction of concretions which naturally form on catheters and the like and for the reduction of bodily concretions and cellular overgrowth.

2. Prior Art

As is appreciated by one of skill in the art there are many conditions occurring within the human body that require introduction of a tube (i.e. stent, catheter, etc.) into the lumen of a bodily organ. Afflictions for which catheters are often used are blockages of the ureter or urethra or vascular or the biliary system. Such blockages can be caused by a traumatic injury, by an illness, etc. that collapse the tube. In any event, action is required to open the lumen and allow excretory fluid to pass therethrough. Commonly, a stent is inserted into the offending vessel or tube to expand it to an open state. The stent is left in situ to prevent another collapse. Stents have been so inserted for a long period of time and historically have had a life span of several weeks or months before encrustation by concretions material and cellular overgrowth begins to occlude the stent or form on the surface of the stent and replacement is required. Concretions such as these obligate the patient to return to the hospital office every few months to endure the painful and risky procedure of undergoing general anesthesia and replacing the stent in order to maintain body function or result in system obstruction. While this is necessary for the patient to remain relatively healthy, it is time consuming and expensive; it is also deleterious to some degree to the body vessel within which the stent is installed. It often occurs that when the stent is removed another cannot be inserted. While stents of the prior art have greatly improved quality of life for people who otherwise would require externally draining tubes or worse, (e.g. poisoned by their own excretory fluids), the need for repeated and frequent visits to the hospital requires alternatives. A longer lasting stent is needed.

With respect to kidney stones, prior art methods for their removal and comminution are also time consuming and expensive since the patient must be attended by the physician for the entire procedure. Often repeat visits are necessary. The art requires advances here as well.

### SUMMARY OF THE INVENTION

The above-discussed and other drawbacks and deficiencies of the prior art in stent use and kidney stone treatment are overcome or alleviated by the piezo-electric stent of the invention.

The invention provides a stent comprising a piezo-electric material preferably in a cylindrical shape and having (one preferred arrangement) concentric cylindrical electrodes on either side of the piezo-electric material. A positive electrode is disposed radially inwardly or radially outwardly and a negative electrode is disposed as the other radially inwardly or radially outwardly placed concentric ring. Upon energization of the piezo-electric material, through the electrodes or other remote method of stimulation, vibration is produced whose frequency is preferably in the range of from about 500 Hz to about 80 kHz and whose wavelength is from about ½ mm to about 1 cm. The vibration reduces and/or prevents the formation of encrustations and concretions on the stent and breaks down those that may have formed. The invention provides for both stationary power sources for non-ambulatory patients and mobile power sources for convenience if the patient is ambulatory.

Another aspect of the invention employs the device of the invention to break down kidney stones in the body. The device, with a portable power supply, advantageously allows a patient to be catheterized and then go home while the piezo-electric stent works on the stone(s) in the kidney and/or ureter to minimize or prevent formation of encrustation on the stent. Vibrations From the stent are transmitted to the stone either directly (if touching) or indirectly through body fluids, and break down the stones(s) by high cycle fatigue, and in some cases by exceeding the yield strength of the stone. Since normal urination can take place through the stent, a major benefit of the invention is that the patient may maintain relatively normal life activities while undergoing treatment for the kidney stone(s). This then avoids the much more costly and time consuming procedures such as lithotripsy, as well as the potentially dangerous procedures of a surgical nature and reduces the frequency with which the stents must be replaced.

The above-discussed and other features and advantages of the present invention will be appreciated and understood by those skilled in the art from the following detailed description and drawings.

### BRIEF DESCRIPTION OF THE DRAWINGS

Referring now to the drawings wherein like elements are numbered alike in the several FIGURES:

FIG. 1 is a schematic perspective view of the stent of the invention having been polarized radially;

FIG. 2 is a schematic perspective view of the stent of the invention having been polarized uniformly perpendicularly to the longitudinal axis of the stent;

FIG. 3 is a schematic view of FIG. 1 wherein insulative layers are added to both exposed electrode surfaces;

FIG. 4 is an alternate embodiment of the invention wherein the electrodes are patterned on the stent to produce spatially varying vibrations along the stent;

FIG. 5 is a schematic perspective view of the stent of the invention in vivo illustrating stones therearound; and

FIG. 6 is a schematic perspective view of FIG. 4 in vivo illustrating stones therearound.

### DETAILED DESCRIPTION OF THE PREFERRED EMBODIMENTS

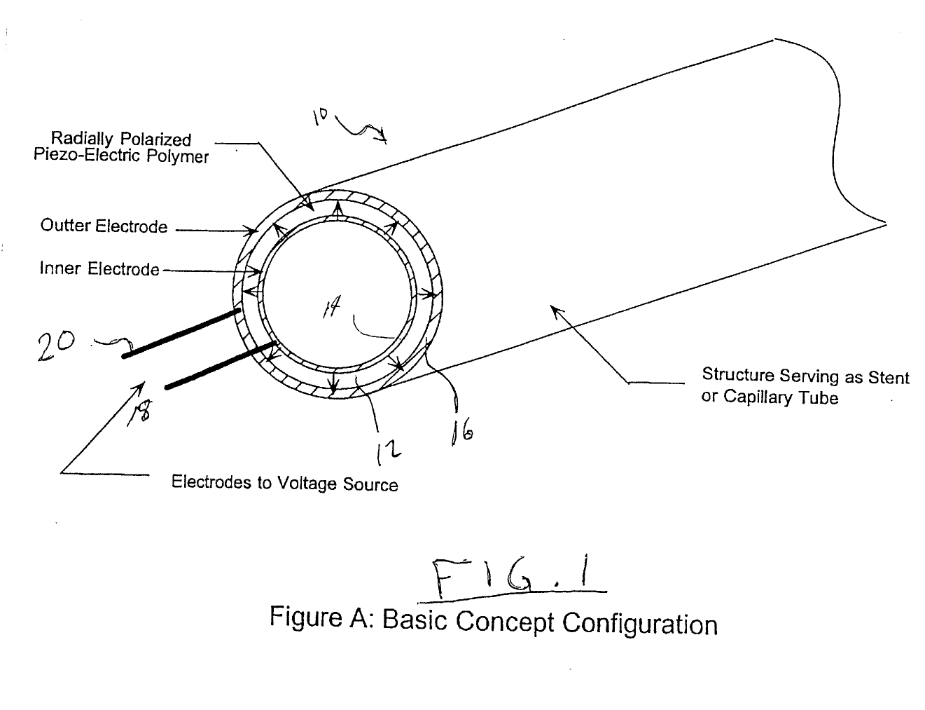

Referring to FIG. 1, a schematic view of the invention 10 will provide to one of skill in the art, a basis for understanding the invention. A piezo-electric material 12 may be selected from known piezo-electric materials including ceramic or polymeric material, polymeric material being preferred due to its flexibility. As will be recognized, a patient will much appreciate a more flexible stent rather than a rigid stent for comfort reasons both during insertion and while the stent is in place. This is due to the tortuous path a stent must take through the urethra, bladder, ureter and to a kidney.

The material 12 selected is permanently polarized at high temperature by applying thereto a high electric field and then cooling the material in that field. Polarity of the material will then be in the direction of the field. Mechanical stress or vibration may then be created in the material by subjecting it to another electrical field. The vibration is the property responsible for the superior results of the present invention.

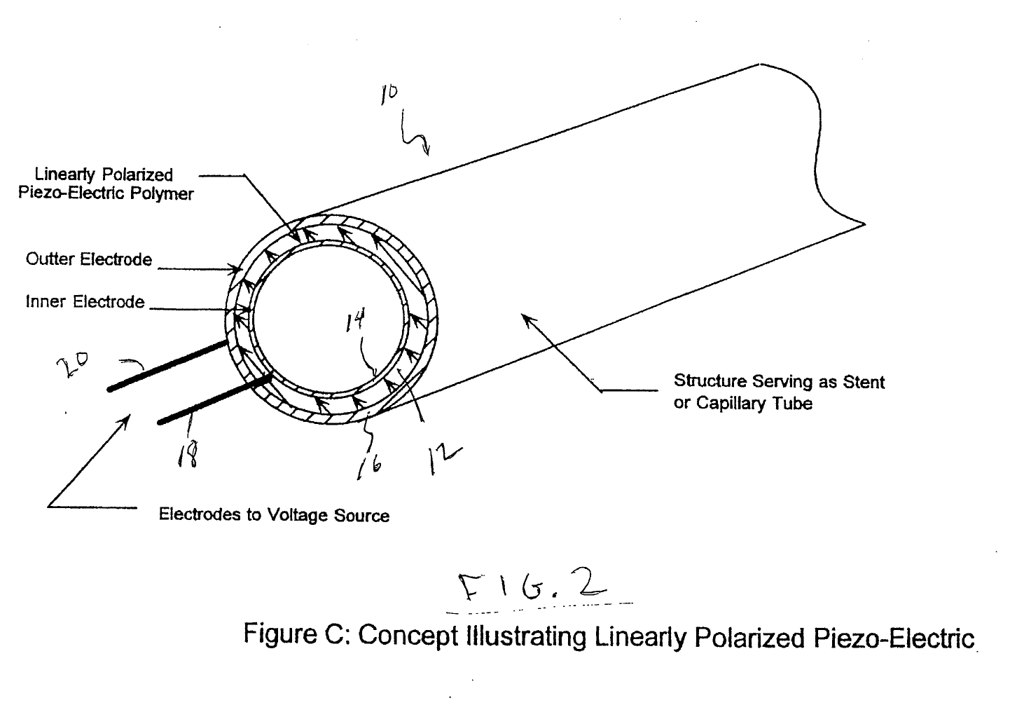

In the preferred embodiments of this disclosure two polarity orientations are preferred. These are radially around the stent (FIG. 1) and uniformly perpendicular to the stent (FIG. 2). The radial configuration provides a uniform radial vibration both at the inner and outer surfaces, thus influencing encrustation formation around and internal to the entire stent or catheter. The transverse polarity excitation produces non-uniform stresses in the material, thus establishing bending movements or non-uniform stresses which cause break up of existing encrustations or inhibit formation of encrustations.

Whether radially or uniformly polarized, the stent 10 provides electrodes 14 and 16 one on either concentric surface of piezo-electric material 12. Energizing electrodes 14 and 16, through schematically illustrated leads 18 and 20, causes an electric field to be created over material 12 which then causes the desired vibration of the stent. It is not material which electrode is positive and which is negative since in either position the electric field is still created and material 12 will vibrate. The vibrations created are preferably defined by a frequency in the range of about 500 Hz to about 80 kHz and a wavelength of from about ½ mm to about 1 cm and by a power supply defined by about 50 volts and about 200 milliamperes. By vibrating the stent, the likelihood of concretions forming on the stent is dramatically reduced. This is because it is very difficult for the microscopic particles to adhere to the stent when it is continually vibrating and because the vibrating surface destroys density gradients in the fluid which are key to encrustation formation. The selected mechanical stress/vibration properties are selected to cause stress within concretions that do accrete, which according to their expected structure and chemical makeup are most likely to cause a breakdown thereof. The breakdown of the concretions is both by vibrations which themselves produce shear stress in the encrustation that is beyond the yield strength thereof or by vibrations that, although the vibrations themselves do not produce shear stresses beyond the yield strength of the encrustations, have such a high cycle that the encrustations become fatigued and breakdown. The vibration of the material prevents the formation of encrustations. Encrustation occurs when a site on the material becomes a location for precipitation. A gradient in the concentration of the offending precipitating ions and species occurs affects the rate of precipitation and agglomeration of the encrusting material at that site. The small wavelength of the imposed vibration of the material destroys these concentration gradients and greatly reduces precipitation and material condensation.

The action of the invention alleviates the formation of encrustations and destroys those that do form with the end result being a stent having significant longevity beyond what the prior art provides. Where a prior art stent might last for 6-12 weeks, the stent of the invention lasts a significantly longer interval before replacement is required. Furthermore the power required by the stent of the invention, identified above, is easily suppliable by a small enough battery or power generator to be carried by the individual within whom the stent is installed. Thus, with the invention, the benefits of longevity are gained, while freedom of movement is not restricted.

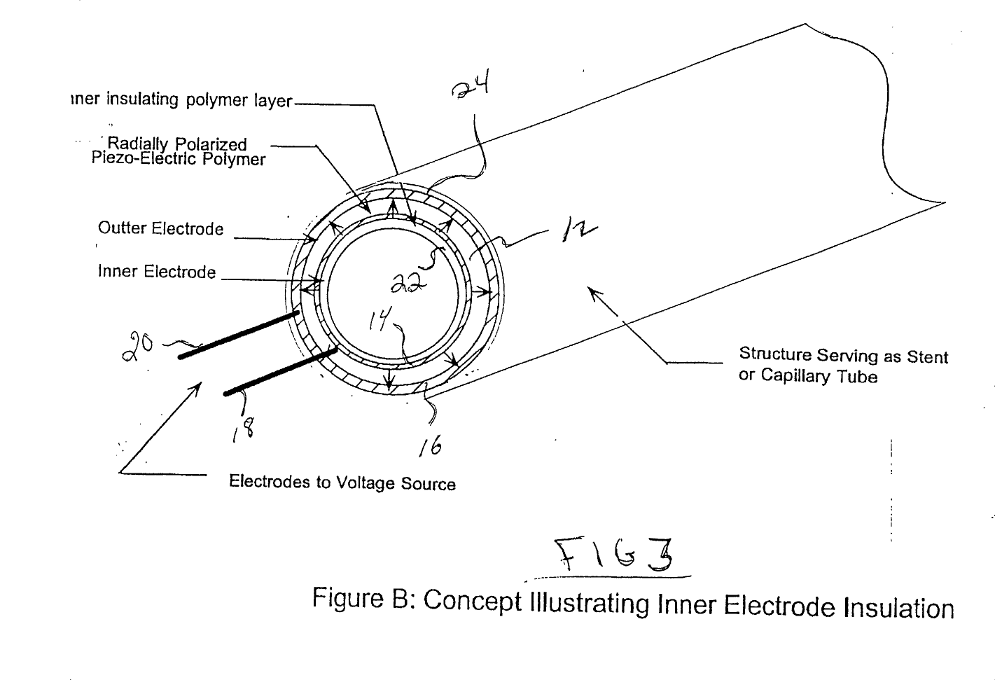

In order to increase the supply of power available to cause the desired piezo-electric activity, and to avoid potential discomfort for the patient, one/or both of the electrodes 14 and 16 is/are coated with an insulative coating 22 and 24 (see FIG. 3). By providing the insulative coating on both electrodes, current will be prevented from flowing into surrounding fluids or tissues which would otherwise reduce the energy available for the piezo-electric activity and might cause some discomfort to the patient including adversely affecting vital functions. As one of skill in the art should understand, only one of the electrodes must be insulated to prevent this loss since this is all that is required to effectively break the potential circuit between the electrodes, current being conducted through body tissues or fluids. It is preferred to simply insulate both electrodes on their exposed surfaces to provide redundancy. In this configuration, if a coating is not complete and could otherwise have bled current off, the insulation on the other electrode will prevent the circuit from forming.

In addition to stationary or portable energy sources for electricity, the invention includes sources of power that couple electro-magnetically to a device implanted in the body, such as a coil implanted with the stent and which is magnetically excited using alternating magnetic fields, thus removing the need for wires. Another embodiment employs a piezoelectric material which is implanted with the stent and which is excited using mechanical vibrations produced externally.

In a preferred embodiment, the electrodes 14 and 16 are conducting polymers or thin coatings of metal or metal alloy material. Electrodes 14 and 16 may be applied to the material 12 in any conventional manner not deleterious to the piezo-electric material.

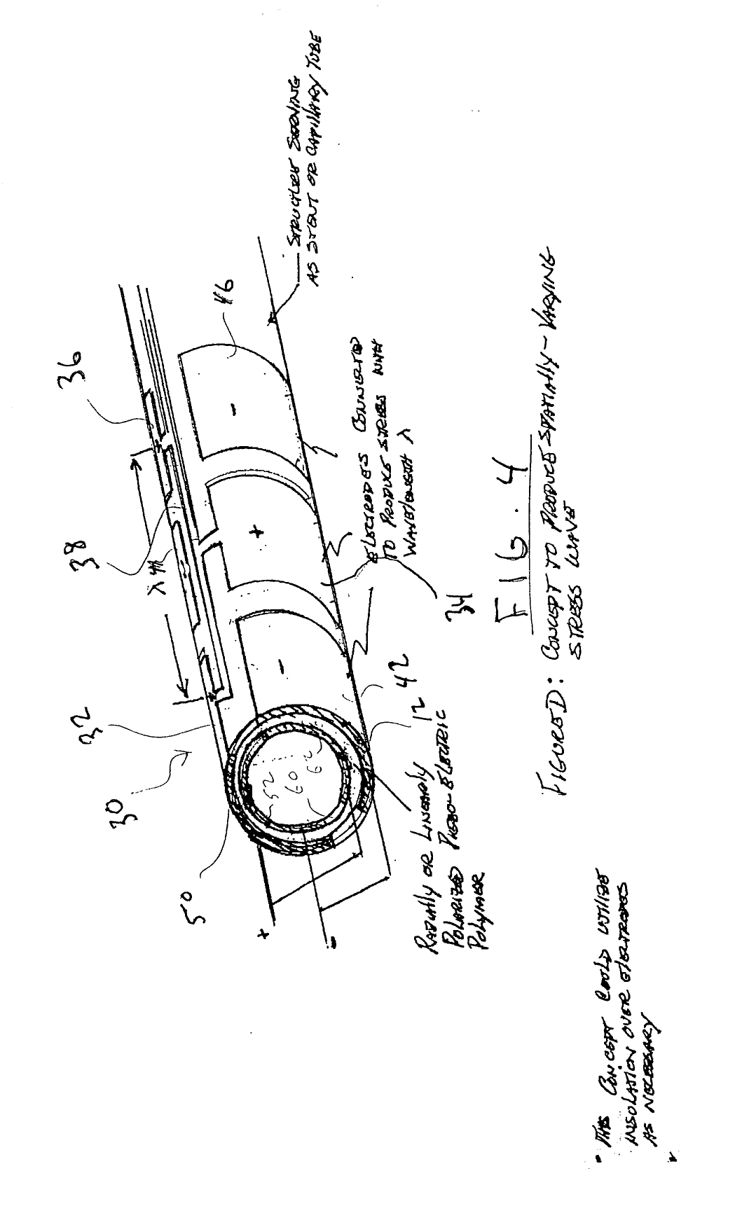

In an alternate embodiment of the invention a series of electrodes of varying polarity are placed in discrete areas of the stent 30. Referring to FIG. 4, an exemplary pattern is illustrated wherein positive electrodes 32, 34 and 36 are fed by trace 38 and negative electrodes 42, 44 and 46 are fed by a mirror image of trace 38 which is not shown. The placement of the electrodes illustrated is on the outer electrical sheath 50 while a similar pattern with opposite polarity is disposed on the inner electrical sheath 60. With respect to sheath 60, only the end electrodes 52 and 62 can be seen and are only visible in cross section. Since electrode 42 is negative, electrode 62 will be positive and because electrode 32 is positive electrode 52 will be negative. The pattern should be understood to one of skill in the art and repeats in like form. Between each inner and outer electrode an electric field is created which causes the piezoelectric material 12 to vibrate. Since the discrete electrode sets (inner/outer) are of alternating polarity and in different places, the electric fields set up in the material 12 are spatially different and cause the vibration to be spatially different. A complex mechanical stress system is caused by the arrangement which accelerates the fatigue of encrustations which form on the stent or stones that are present near the stent. Thus the stent of the invention solves the prior art need by providing a life span significantly greater then what has heretofore been known.

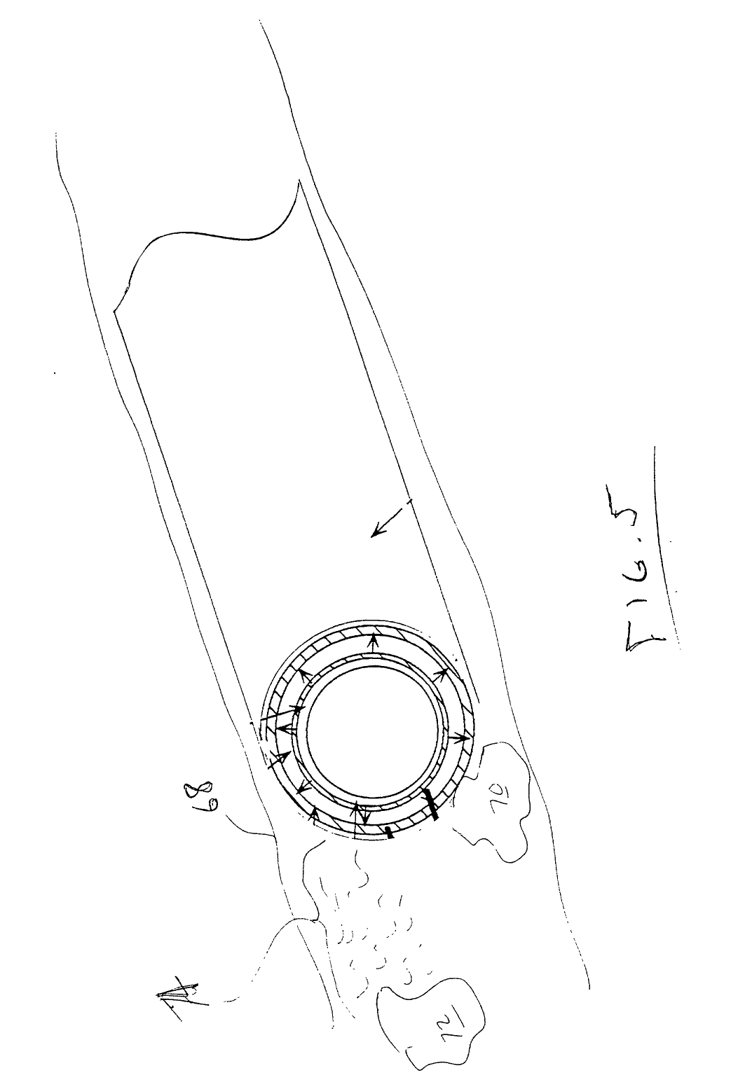

In another aspect of the invention, it finds use as a kidney stone reducer that simultaneously avoids the more time consuming and expensive procedures currently employed. The reduction of kidney stones is an additional benefit of the employment of the stent of the invention for the normal and common purpose of expanding the ureter, yet is so effective, the invention is also employed for this purpose alone. While the energized stent is reducing the formation of and breaking up of encrustations the vibrations are also propagated through fluid and through tissue to some degree to stones located near the placement of the stent. The vibrations tend to stress the stone cyclically and cause it to break down. Moreover, where a stone has moved into contact with the stent, the vibrations will be directly transmitted to the stone resulting in its destruction. The reduction of stones is accomplished by either the radial polarized stent or the uniformly polarized stent energized in any one of the above-described configurations. Referring to FIG. 5, stent 10 is illustrated in ureter 68, one stone 70 is illustrated in contact with stent 10 while another stone 72 is not in contact with the stent 10. Stone 70 will break down faster but stone 72 will also be eroded by the propagated vibrations shown schematically by wavy lines 74.

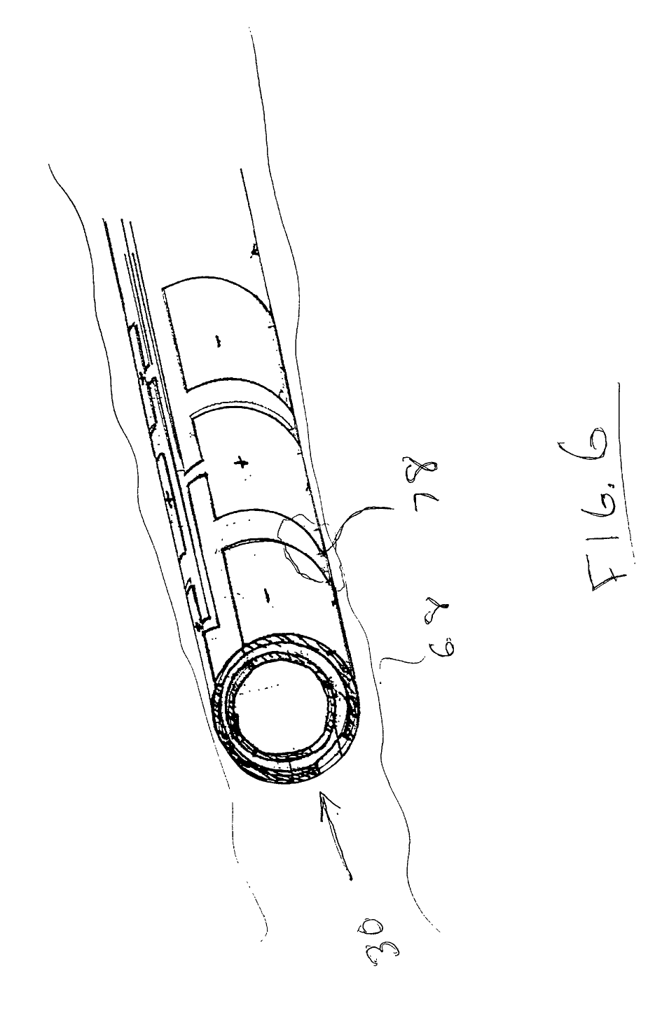

Referring to FIG. 6 the reader will recognize that the embodiment of FIG. 4 is illustrated within ureter 68 and with stone 78 wedged between ureter 68 and stent 30. In this embodiment the stone is reduced as-it was in the previous discussion, however the speed with which the stone is reduced in size is accelerated in this embodiment by the spatially different vibrations created. Stone 78 is illustrated spanning the electrodes (42 and 34). By so doing, different vibrations are simultaneously introduced into the stone. This is a severe stress situation and causes the stone to breakdown very quickly.

A major benefit of the employment of a stent of the invention to destroy and pass kidney stones, as opposed to lithotripsy or other common means is that the patient visits the doctor once and goes home with a portable power source. This is possible with the stent of the invention because the patient may urinate through and around the stent. Prior art piezoelectric stone reducing apparatus provide no such benefit. The patient would need to be attended by the physician operating the piezo-electric device until the stone was eliminated. In the invention the patient is catheterized and goes home for the stent to work. After a given period of time another visit will result in removal of the stent and passing of the stone. The patient need not spend a large amount of time at the doctor's office and need not be sent to a remote and expensive lithotripsy facility. Thus there is a savings to the patient in time, money and aggravation.

It should be understood that although cylindrical stents are shown and described the invention could be produced from other geometric shapes without departing from the spirit and scope of the invention. More specifically, the invention extends to non-tubular shapes for the piezo-electric material as well. In vivo dispatch of piezo-electric devices to reduce the formation or size of concretions has not heretofore been known. Moreover the electrode placement is exemplary, other placements being equally effective.

While preferred embodiments have been shown and described, various modifications and substitutions may be made thereto without departing from the spirit and scope of the invention. Accordingly, it is to be understood that the present invention has been described by way of illustration and not limitation.

What is claimed is:

1. A piezo-electric in vivo insertable device comprising:
a) a piezo-electric material in a tubular shape having an interior surface and an exterior surface;
b) at least one inside electrode on said interior surface of said piezo-electric material;
c) at least one outside electrode on said exterior surface of said piezoelectric material.

2. A piezo-electric in vivo insertable device as claimed in claim 1 wherein said at least one inside electrode covers said interior surface of said piezo-electric material.

3. A piezo-electric in vivo insertable device as claimed in claim 1 wherein said at least one inside electrode is a plurality of electrodes having inconsistent polarity among the plurality of electrodes.

4. A piezo-electric in vivo insertable device as claimed in claim 1 wherein said at least one outside electrode covers said exterior surface of said piezo-electric material.

5. A piezo-electric in vivo insertable device as claimed in claim 3 wherein said at least one outside electrode is a plurality of electrodes having inconsistent polarity among the plurality of electrodes.

6. A piezo-electric in vivo insertable device as claimed in claim 5 wherein said plurality of inside electrodes are each paired with one of said plurality of outside electrodes.

7. A piezo-electric in vivo insertable device as claimed in claim 1 wherein said piezo-electric material is radially polarized.

8. A piezo-electric in vivo insertable device as claimed in claim 1 wherein said piezo-electric material is uniformly perpendicularly polarized.

9. A piezo-electric in vivo insertable device as claimed in claim 1 wherein said at least one of said at last one inside electrode and said at least one outside electrode is an insulated electrode.

10. A piezo-electric in vivo insertable device as claimed in claim 9 wherein said insulated electrode is a positive electrode.

11. A piezo-electric in vivo insertable device as claimed in claim 1 wherein said at least one inside electrode and said at least one outside electrode are insulated electrodes.

12. A piezo-electric in vivo insertable device as claimed in claim 1 wherein said insertable device is a stent.

13. A piezo-electric in vivo insertable device as claimed in claim 1 wherein said device is a capillary tube.

14. A piezo-electric in vivo insertable device as claimed in claim 1 wherein said device vibrates at a frequency in the range of about 500 Hz to about 80 kHz and with a wavelength of in the range of about ½ mm to about 1 cm.

15. A piezo-electric in vivo insertable device as claimed in claim 1 wherein said device further includes a power source.

16. A piezo-electric in vivo insertable device as claimed in claim 15 wherein said source is mobile.

17. A piezo-electric in vivo insertable device as claimed in claim 15 wherein said source is stationary.

18. A method for reducing encrustation of an in vivo insertable tube comprising;
a) inserting the device of claim 1 into a target position in a body;
b) supplying power to said tube causing said tube to vibrate.

19. A method for reducing encrustation of an in vivo insertable tube as claimed in claim 18 wherein said power is supplied by a stationary supplier.

20. A method for reducing encrustation of an in vivo insertable tube as claimed in claim 18 wherein said power is supplied by a mobile supplier.

21. A method for treating bodily concretions in vivo comprising;
a) inserting the device of claim 1 into a target position in a body;
b) causing said device to vibrate;
c) leaving said device in place in vivo for a period of time.

22. A method for treating bodily concretions in vivo as claimed in claim 21 wherein said method further includes removing said tube from the body.

23. A method for treating bodily concretions in vivo as claimed in claim 21 wherein said vibrations are transmitted directly to a stone in contact with said device.

24. A method for treating bodily concretions in vivo as claimed in claim 21 wherein said vibrations are transmitted through bodily fluid to a stone spaced from said device.
